(19) United States
(12) Patent Application Publication
(10) Pub. No.: US 2009/0240208 A1
(43) Pub. Date: Sep. 24, 2009

(54) Microparticle delivery syringe and needle for placing particle suspensions and removing vehicle fluid

(75) Inventor: Benjamin David Cowan, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/051,419

(22) Filed: Mar. 19, 2008

(51) Int. Cl. A61M 5/19; A61M 5/31

(52) U.S. Cl. 604/190; 604/191

(57) ABSTRACT

A syringe having two or more chambers having a needle defining two or more lumens for injecting a microparticle suspension into a desired space, the lumens being in fluid communication with respective chambers. One of the chambers has the microparticle suspension disposed therein while the other chamber is empty. The lumen in fluid communication with the empty chamber has a filter element disposed therein such that the suspension fluid may be withdrawn from the space into the empty chamber without withdrawing the microparticles.

### FIELD OF THE INVENTION

The present invention relates to a microparticle delivery device, and, more specifically, to a dual-chambered syringe having a bifurcated needle with lumens in fluid communication with respective chambers. The device allows the injection of a suspension of microparticles and the subsequent removal of the fluid delivery media.

### BACKGROUND OF THE INVENTION

Microparticles are generally defined as being particles between 0.1 to 100 microns in size and can be formed from a variety of materials, including proteins, polymers, polysaccharides and combinations thereof. It is known in the art to use microparticles for a variety of purposes, including use as carriers of active pharmaceutical substances. Because of certain requirements imposed upon the delivery of pharmaceuticals via microparticles, it is desirable that the microparticles have a substantially spherical shape and a narrow size distribution. Microparticles used for such purposes are often delivered by injection through a syringe. When delivered by this route, the microparticles may be in suspension in an aqueous solution.

Microparticles are typically suspended in solution for injection into a target space, which may be, for example, an anatomical space in a patient (human or otherwise), or other confined spaces, such as refillable implantable pumps, venous access ports and the like. Such target spaces may be small and therefore may limit the amount of microparticles that can be delivered. It would be desirable to be able to remove the suspension fluid after delivering the microparticle suspension into the target space so that another injection could be administered until the space is filled with the maximum or desired amount of microparticles. Hence, it would be desirable to have a device that allows the removal of the suspension fluid, without removing the therapeutic microparticles. This would allow room in the target space for an additional injection of suspended microparticles.

### SUMMARY OF THE INVENTION

The objectives of the invention can be realized by administrating a microparticle suspension using a bifurcated syringe device having two chambers and a needle with two lumens, one lumen being fluidly connected to each chamber. One chamber of the device is filled with the microparticle suspension, while the other chamber remains empty or primed with a suitable fluid. Pushing the plunger of the chamber containing the microparticle suspension injects the suspension through one lumen of the needle and into the target space. Thereafter, a reverse motion of the plunger in the other chamber creates a negative pressure that pulls the suspension fluid from the target space through a filter disposed in the second lumen, the filter having pores smaller than the diameter of the microparticles which were injected. The microparticles will therefore remain in place within the target space when the fluid is removed. The removed fluid is contained in the second chamber, separate from the first chamber holding the microparticle suspension.

Once a volume has been withdrawn, which may be, for example, equal to or less than the fluid volume of the microparticle suspension, another injection of the suspension can be administered and the process repeated as desired until the maximum or target amount of microparticles have been delivered. The total volume of the multi-step administration delivered to the target space may be the additive volume of the accumulated microparticles in-vivo and the volume of the final injection of the microparticle suspension. This can be readily assessed by the operator as the volume expelled from the microparticle suspension chamber minus the volume in the withdrawn fluid chamber.

In a second embodiment, a trocar guide channel can remain in place, while separate injecting and expelling syringes and needles are interchanged for injection and withdrawal steps.

### DESCRIPTION OF THE DRAWINGS

FIG. 1 is cross sectional view of an embodiment device.

FIG. 2A is a side view of the device of FIG. 1 showing a bifurcated needle and a filter disposed in one lumen of the needle.

FIG. 2B shows a lower end of the bifurcated needle of FIG. 2 in perspective view with a filter disposed in one lumen of the needle.

FIG. 3A shows a side view of the syringe of FIG. 1.

FIG. 3B illustrates a bifurcated needle showing two lumens without a connecting mechanism used to connect the needle to a syringe.

FIG. 3C illustrates the lower portion of an embodiment syringe, showing a bifurcated outlet.

FIG. 3D shows the needle of FIG. 3B having a connecting mechanism thereon.

FIG. 4 is a side view of another embodiment needle.

FIGS. 5A-5C are side views of a distal end of a further embodiment needle.

FIGS. 6A-6C are side views of a distal end of a yet another embodiment needle.

FIGS. 7A-7D are views of a distal end of still another embodiment needle.

### DETAILED DESCRIPTION OF THE INVENTION

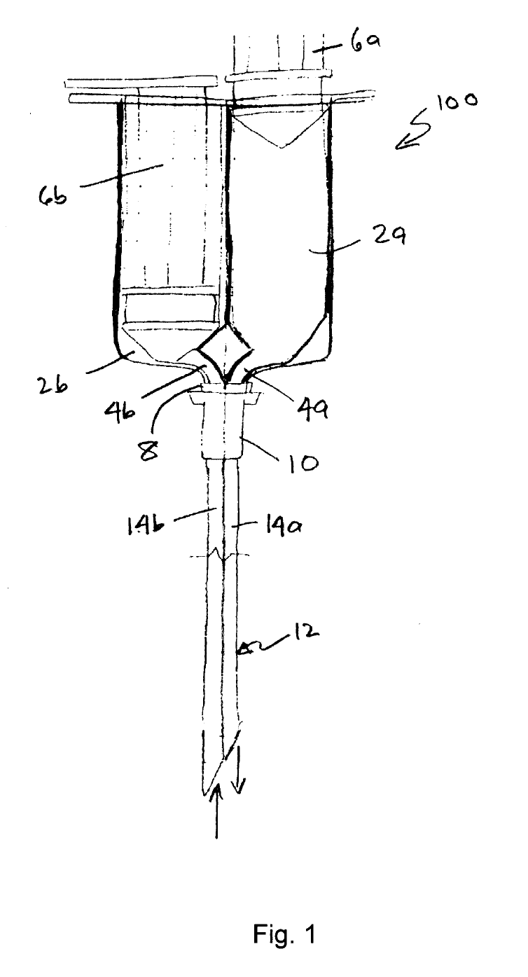

An embodiment bifurcated syringe 100 is shown in a cross-sectional view in FIG. 1. The body 29 of the syringe includes two or more chambers. FIG. 1 shows an embodiment of the syringe 100 having two chambers, labeled 2a and 2b. One chamber may hold a microparticle suspension and the second chamber may hold the suspension fluid after removal from the target space. As previously indicated, a target space may be an anatomical space within a patient (which may be human or otherwise), or a space within a pump, depot, access port or the like. In FIG. 1, syringe 100 is shown with plunger 6a in the fully proximal position and plunger 6b in the fully distal position. Preferably the microparticle suspension would be loaded in chamber 2a to be injected into the target space by pushing plunger 6a distally, thereby forcing the microparticle suspension out of chamber 2a through channel 4a.

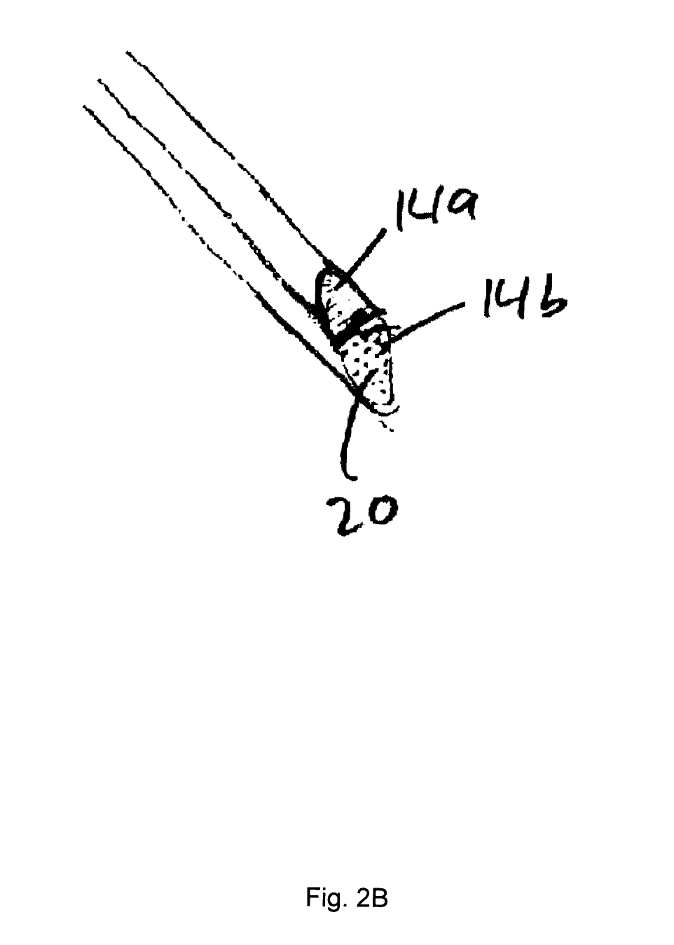

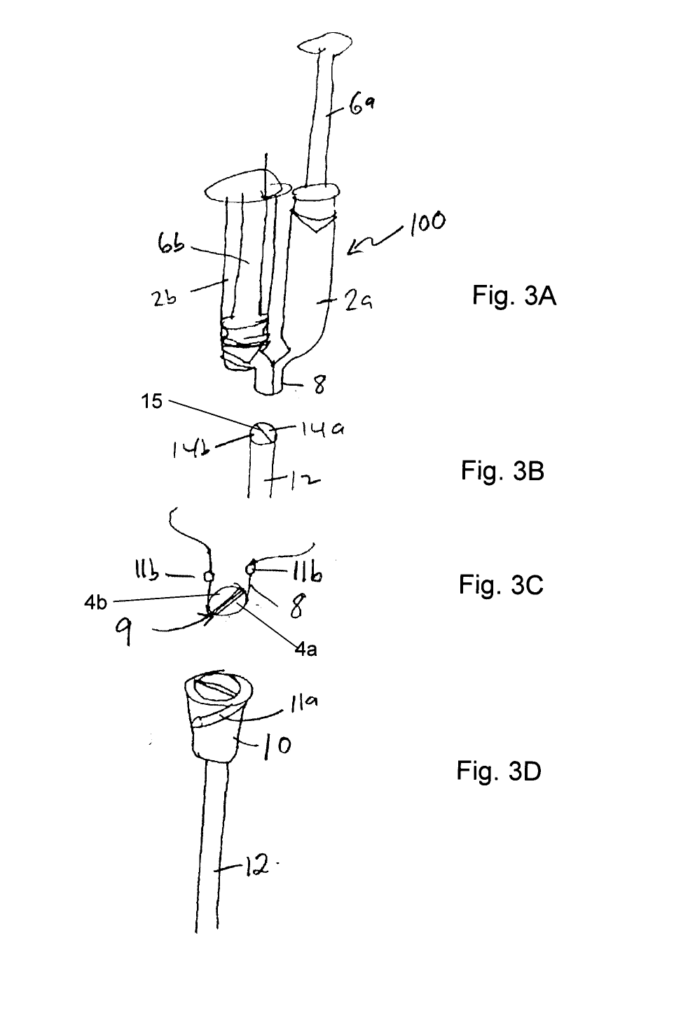

Both chambers 2a, 2b are fluidly connected to needle 12 through respective channels, labeled 4a and 4b, which are in fluid communication with the chambers 2a and 2b and needle 12. As can be seen in FIG. 3C, lower portion 8 of syringe 100 contains a bifurcated outlet having two or more channels 4a, 4b separated by a partition 9. FIG. 3C shows outlet 8 of the two chamber syringe 100 pictured in FIG. 1, with one half of outlet 8 communicating with chamber 2a through channel 4a and the other half of outlet 8 communicating with chamber 2b through channel 4b.

FIG. 3A is a side view of the syringe 100. Note that the respective chambers 2a and 2b may share a common wall or may be completely independent of each other. In addition, they may be connected by a bracket or other suitable forms of attachment (not shown) to keep them in relative position with respect to each other. Plungers 6a and 6b, however, are ideally able to move independently of each other.

The body of syringe 100 may be formed from any known material of which syringes of the prior art are normally manufactured, preferably plastic or glass. Plungers 6a and 6b may be standard syringe plungers as would be found in single chamber syringes well known in the art.

FIG. 3B shows the upper end of a bifurcated needle 12, showing two lumens 14a and 14b, which are in fluid communication with chambers 2a and 2b respectively. FIG. 3D shows the same bifurcated needle 12 having a connecting mechanism 10 on the upper end thereof for connecting with syringe 100. FIG. 3C shows the mating portion 11b on the syringe 100 for the connector 10. One or more protrusions 11b on either side of outlet 8 of syringe 100 ride in corresponding channels 11a, shown in FIG. 3D, located in connector 10 of needle 12, and serve to ensure that lumens 14a and 14b align with channels 4a and 4b respectively in outlet 8. The wall 15 between lumens 14a and 14b ideally lines up with partition 9 between channels 4a and 4b as well. Connector 10 may employ, for example, a gasket or the like to ensure between the needle 12 and the outlet 8, as well as to ensure that the fluidic pathways defined by channels 4a, 4b and their corresponding lumens 14a, 14b remain isolated from each other. Alternatively, or in conjunction with a gasket, a male-female connection could be employed between the wall 15 of needle 12 and the partition 9 of outlet 8.

Needle 12 connects to syringe 100 by a slight turning motion which engages one or more protrusions 11b with the corresponding threads 11a. Any other prior knowledge known to one of skill in the art may be used to secure needle 12 to syringe 100 as long as the individual lumens 14a, 14b within needle 12 line up with their corresponding channels 4a and 4b in syringe 100 to form isolated fluidic pathways for the transfer of the suspension.

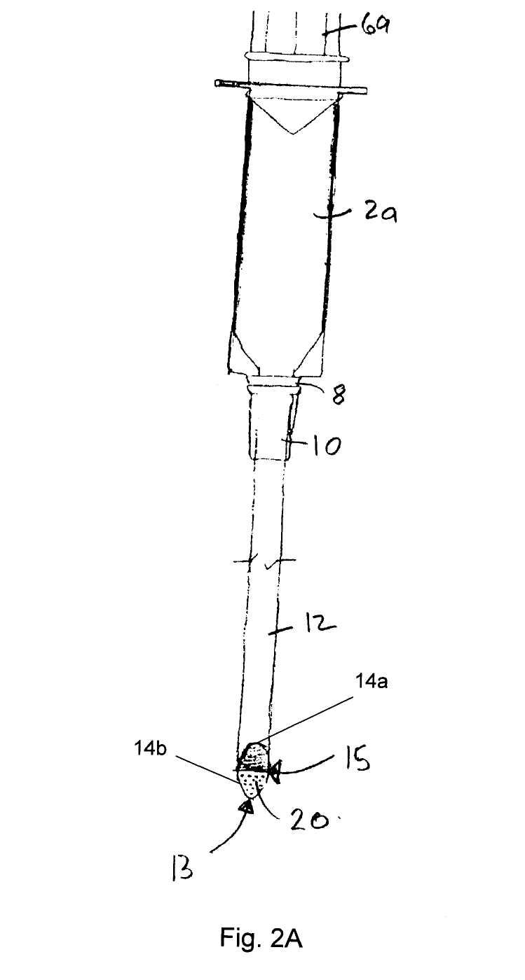

FIG. 2A is a side view of syringe 100 showing one chamber 2a and also showing the distal end of needle 12 with the two lumens 14a and 14b being clearly visible. Preferably, the distal end of needle 12 is cut on a taper having an oblique angle, which may form an oval-shaped cross-section, thereby forming a sharp point 13 capable of piercing the skin of the patient, the surface of a device or the like. It is preferable that the tapered cut in the end of the needle 12 and the opening created thereby be bisected by the wall 15 which divides lumens 14a and 14b, preferably leaving one lumen 14b disposed at the lower, most distal, end of the opening and one lumen 14a disposed at the upper, slightly more proximal, end of the opening, as shown in FIGS. 2A and 2B.

It is preferable, although not required, that the shorter lumen (i.e. the lumen disposed on the upper portion of the opening, labeled 14a in FIG. 2B), be the one through which the micro-particle suspension is injected into the target space.

Filter 20 is located within the second lumen 14b and is used for extraction of the suspension fluid from the target space. Filter element 20 includes a plurality of pores that permit fluid to pass through filter element 20. It is desirable, however, that the pores of filter element 20 be smaller than the average diameter of the microparticles to avoid removing the microparticles from the target space when the suspension fluid is removed. In certain embodiments, the filter element 20 may actually be provided by a plurality of holes in and around the tip 13 of the needle 12. In such embodiments the lumen 14b may be closed at its most distal end; fluidic communication of lumen 14b with the target space may be provided by a plurality of holes in lumen 14b, both at the tip 13 of the needle and optionally along the sidewalls of lumen 14b. The holes are sized to prevent the inflow of microparticles into lumen 14b, and may be formed by any suitable process, such as machining, etching or the like. In other embodiments the filter element 20 may be a paper insert or the like inserted into the lumen 14b and positioned near the tip 13 of the needle 12. In specific embodiments the filter 20 is preferably flush with the oval-shaped cross-sectional area of the needle opening, and hence flush with the distal opening of the lumen 14b.

Other configurations of the distal end 13 of needle 12 are possible. For instance, wall 15 which divides lumens 14a and 14b within needle 12 may be at any angle within the opening of the needle, thus providing different shaped openings for each of lumens 14a and 14b. It is also possible to cut the end of needle 12 at different angles, which may change the relative area of the openings of respective lumens 14a and 14b. Additionally, it is also possible that barrier 15 separating lumens 14a and 14b be off-center within the needle 12, thus creating one lumen with a larger volume than the other lumen, which may be used, for example, to accommodate the filter element within the larger lumen.

In operation, plunger 6a is utilized in much the same manner as a typical lumen syringe and needle; whereby plunger 6a is proximally advanced to create a negative pressure within chamber 2a that draws a suspension comprising microparticles and a carrier fluid into chamber 2a via, for example, lumen 14a. Once chamber 2a is loaded, the needle 12 may be positioned so that the distal end 13 is in or near the target space, which may be an anatomical space within a patient or, for example, another preferred therapeutic space with a confined volume. The plunger 6a is then advanced distally, thereby forcing the microparticle suspension out of chamber 2a, through channel 4a and into lumen 14a of needle 12, and ultimately into the target space. It may be desirable to inject only a portion of the microparticle suspension from chamber 2a into the target space.

After the initial injection of the microparticle suspension, the suspension fluid is preferably withdrawn by creating a negative pressure in chamber 2b by pulling proximally on plunger 6b, which will draw fluid in the target space through lumen 14b, through channel 4b and into chamber 2b. The directions of preferred fluidic flows are shown near the distal end of needle 12 in FIG. 1. As previously discussed, filter 20, shown in FIG. 2, is disposed within lumen 14b. The filter 20 has pores that are ideally smaller than the average size of the microparticles which were injected from chamber 2a, thereby preventing the microparticles from being drawn back into chamber 2b with the suspension fluid. It may be advantageous to periodically reverse the fluidic flow along lumen 14b to flush microparticles from the filter 20 and then re-performing fluidic withdraw from the target space.

Once a volume of suspension fluid is withdrawn into chamber 2b, additional volumes of the microparticle suspension may be injected from chamber 2a, and the process may be repeated several times until chamber 2a is empty or the desired amount of microparticles have been deposited in the target space.

In an alternate embodiment of the invention, a trocar can remain in place while separate injecting and withdrawing syringes and needles are interchanged. In this embodiment, the syringe used for injection of the micro-particle suspension would be a standard syringe, while the syringe used for the withdrawal of the suspension fluid is a standard syringe having a filter disposed in the lumen of its needle.

Once the desired volume of microparticles are in place in the target space, the needle is withdrawn. A certain volume of suspension fluid may also be left in place by injecting the microparticle suspension and not withdrawing the last volume of suspension fluid which was injected.

Needle 12 may be of a size necessary to accommodate at least two lumens suitably sized to inject the microparticle suspension and withdraw the fluid.

In other embodiments, multi-chamber syringes having more than two chambers may be utilized with needles having two or more lumens, such as would be the case if it was desired to mix two microparticle suspensions in the anatomical space. In such cases there may be only one lumen of the corresponding needle which is utilized for withdrawal and which therefore is equipped with a filtering element.

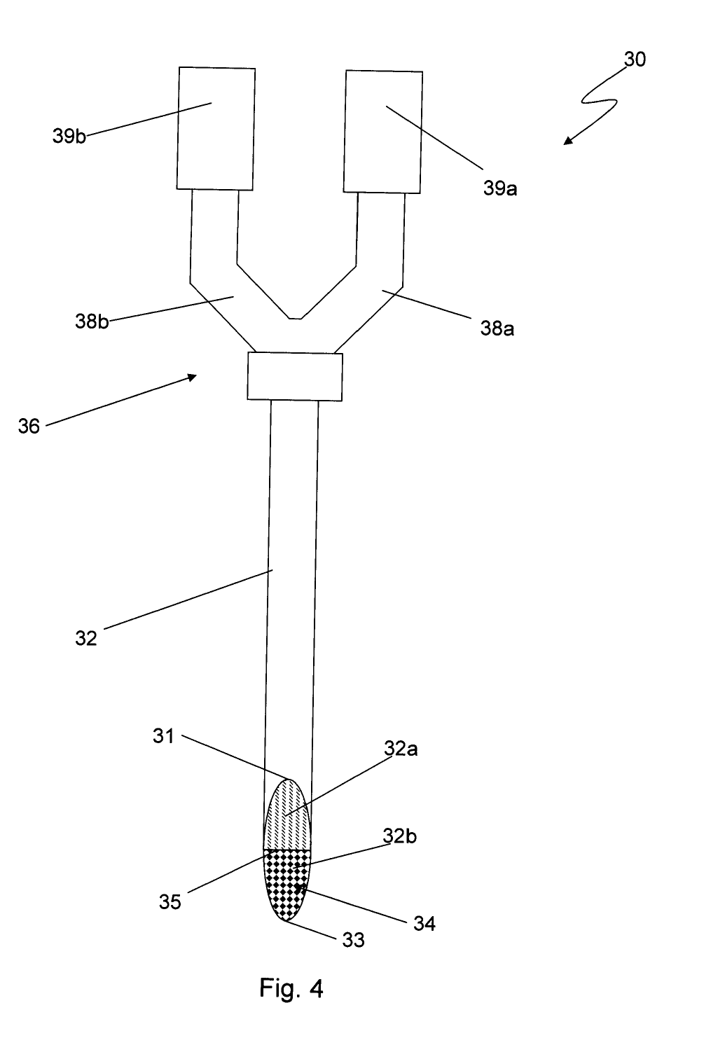

FIG. 4 is a side view of an embodiment multiple-lumen needle 30. The needle 30 includes a shaft 32 comprising a first lumen 32a and a second lumen 32b that are fluidly isolated from each other along the length of the shaft 30 by wall 35. The distal end of shaft 32 preferably has an angular cut providing an oval-shaped cross-section 31 that yields a sharp point 33 at the most distal end of needle 30. Either one of the lumens 32a, 32b includes a filter 34 that is disposed at the distal end of the lumen 32a, 32b, and which is preferably positioned near cross-section 31. As discussed earlier, the filter 34 is preferably disposed within the longer lumen 32b of the two lumens 32a, 32b. The filter is preferably positioned so that its most distal surface flush with the distal opening of the shaft 32 so that there is little or no movement of microparticles along the lumen 32b. However, it will be appreciated that when disposing the filter 34 at the distal end of lumen 32b, the filter 34 may be slightly proximally advanced along the lumen 32b. The amount of such proximal displacement of the filter 34 along the lumen 32b will be a function of how much corresponding loss of the microparticles a practitioner is willing to accept. Hence, disposing of the filter 34 at the distal end of shaft 32 should be understood to include such additional proximal displacements.

The proximal end of shaft 32 includes a sub-connector 36 adapted to fluidly connect the shaft 32 to two corresponding connectors 39a, 39b for standard syringes (not shown) so that each syringe is fluidly connected to a corresponding lumen 32a, 32b. The connector 36 has a first channel 38a that is exclusively fluidly connected to first lumen 32a, and a second channel 38b that is exclusively fluidly connected to second lumen 32b. The sub-connector may mate with wall 35 to ensure the fluidic isolation of the channels 38a, 38b and their corresponding lumens 32a, 32b. The proximal end of each shaft 38a, 38b terminates in a corresponding connector 39a, 39b, that is adapted to connect to a respective syringe. Any suitable connector 39a, 39b may be employed. A non-limiting example of such a connector 39a, 39b includes a luer-lock.

It will be appreciated that the needle 30 may have more than two lumens 32a, 32b, and independently may have more than two channels 38a, 38b and connectors 39a, 39b. Typically there will be a one-to-one correspondence between lumens, channels and connectors. However, in the event that there are more channels 38 than lumens within shaft 32, sub-connector 36 may route two or more channels 38 to a single lumen within shaft 32.

In use, a practitioner may ready two syringes, a first loaded with a microparticle solution and the second empty or primed to receive the microparticle carrier solution. The first syringe is fluidly connected to first connector 39a, and the second syringe is fluidly connected to second connector 39b. The steps discussed above may then be performed, with first lumen 32a dispensing the microparticle solution into the target region, while second lumen 32b removes the microparticle carrier solution from the target region, with filter 34 preventing the uptake of the microparticles into the second lumen 32b.

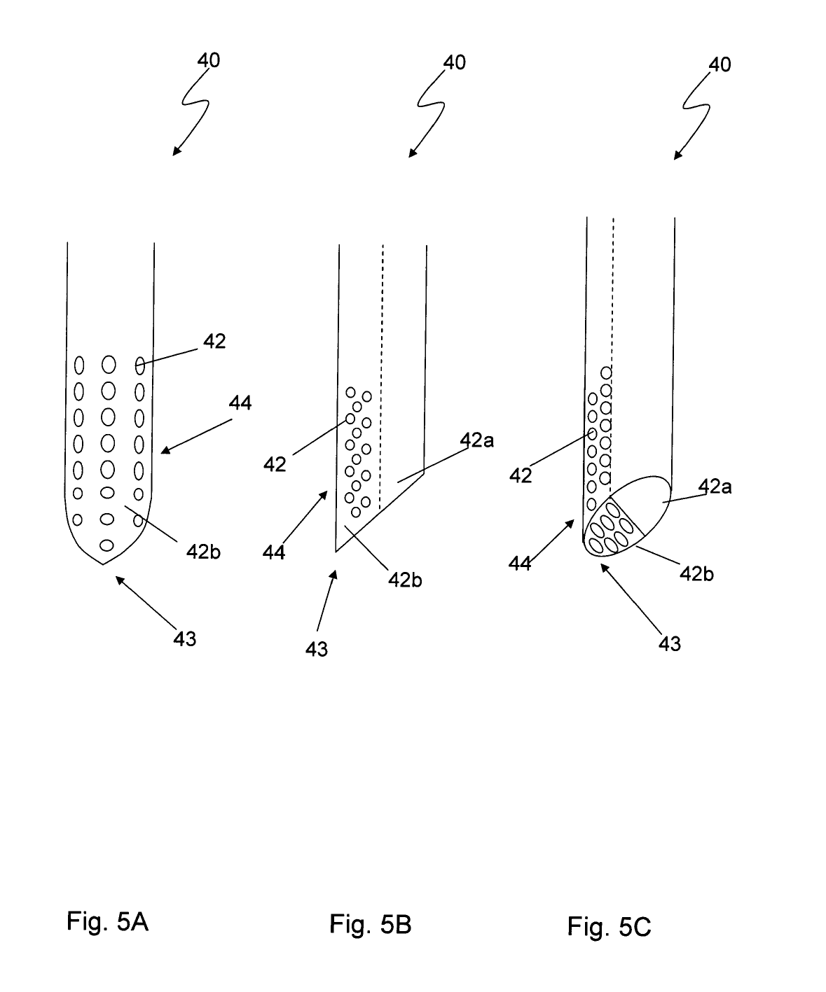

Various methods may be employed to increase the surface area that the filter 34 presents to the target space. FIGS. 5A-5C show different views of a distal end of an embodiment needle 40. Needle 40 has two lumens 42a, 42b, the longer of which 42b includes a filter 44 formed by a plurality of holes 42 in the sidewalls of lumen 42b that present to the target space. One of these sidewall includes the cross-sectional face of lumen 42b that is closed at the most distal tip 43 but is fluidly connected with the target region by way of the holes 42. The holes 42 also extend proximally along the shaft of needle 40 to effectively increase the active surface area of filter 44. The holes may be formed by, for example, machining, etching or any other suitable method. In some embodiments, the holes 42 are present only on the cross-sectional face of lumen 42b at the tip 43, while in other embodiments the holes 42 are present only along the shaft of needle 40.

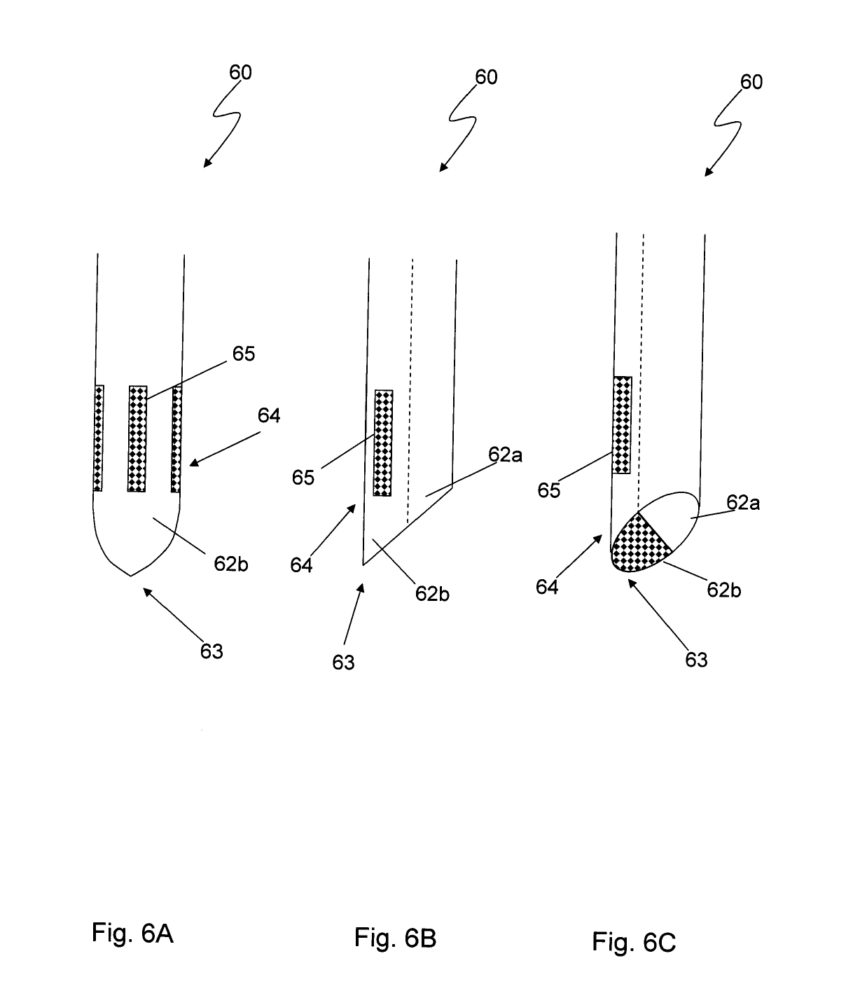

FIGS. 6A-6C show different views of a distal end of another embodiment needle 60. The needle 60 has two lumens 62a, 62b, into the longer of which 62b is disposed a filter 64. The filter 64 may be, for example, an insert which is positioned so as to be near or flush with the cross-sectional opening of lumen 62b at most distal tip 63, and extends proximally along the shaft of needle 60. The filter insert 64 may be made from, for example, a suitable filter paper, cloth or the like. Lumen 62b may also include one or more openings 65 in its sidewall along the shaft of needle 60. The opening or openings 65 are spaced a predetermined distance proximally from tip 63. Filter insert 64 covers openings 65, thus presenting a larger surface area to the target area. Alternatively, a plurality of filter inserts 64 may be used to each respectively cover an opening 65 in lumen 62b, including the cross-sectional opening at the tip 63.

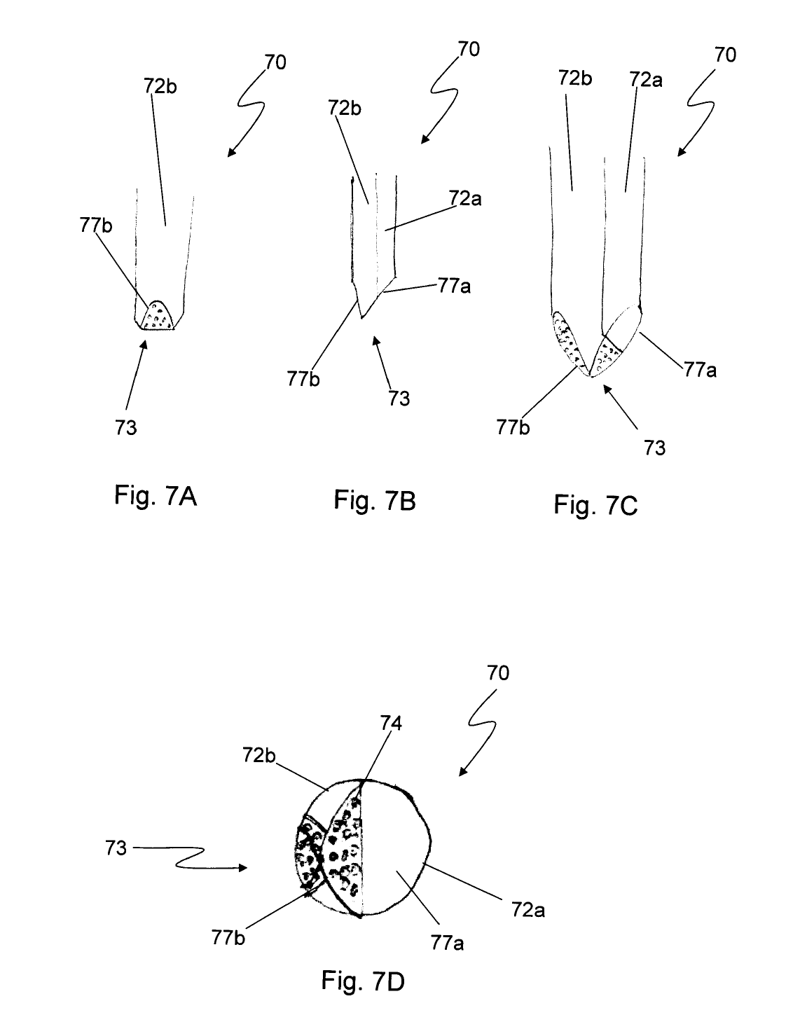

Yet another embodiment needle 70 is shown in FIGS. 7A to 7D, in which FIGS. 7A-7C show various side views of the distal end of needle 70, and FIG. 7D shows a top view of the distal tip 73 of needle 70. The needle 70 uses a plurality of bevels 77a, 77b to increase the effective surface area of filter 74, which is disposed on the longer 72b of two lumens 72a, 72b. As in the embodiment needle 40, filter 74 of needle 70 is provided by a plurality of holes 74 present in the sidewalls of lumen 72b. For the embodiment needle 70, the holes 74 are present only on the surfaces of the cross-sectional areas presented by the bevels 77a, 77b on lumen 72b. Hence, only one of the bevels 77a is present across lumen 72a, whereas lumen 72b which has the filter 74 is crossed by both bevels 77a, 77b. In yet other embodiments, a filter insert, as in embodiment needle 60, may be used instead of the holes 74.

Note that the specifics embodiments are described in an exemplary manner and are not intended to limit the invention. In particular, syringes and needles manufactured of any acceptable material are contemplated to be within the scope of the invention, as are syringes and needles having varying design configurations and numbers of chambers and lumens. The scope of the invention is therefore defined in the claims which follow.

1. A microparticle delivery syringe for delivering a microparticle suspension to a target space, the microparticle delivery syringe comprising:
a. a first chamber;
b. a second chamber fluidly separated from said first chamber;
c. a needle having a shaft with at least two lumens, wherein one of said lumens is in fluid communication with said first chamber and wherein the other of said lumens is in fluid communication with said second chamber; and
d. a filter element disposed at a distal end of the shaft in one of said lumens, the filter element fluidically disposed between the target space and only the said one of said lumens.

2. The syringe of claim 1 further comprising a first plunger slidably disposed in said first chamber and a second plunger slidably disposed in said second chamber.

3. The syringe of claim 2 wherein said plungers can be activated independently of each other to create a positive or negative pressure within their respective chambers.

4. (canceled)

5. The syringe of claim 1, wherein said needle defines an angular cut on the distal end thereof forming an oval-shaped cross-section.

6. The syringe of claim 5 wherein said first and second lumens are of different lengths due to their position within said needle with respect to said oval-shaped cross-section.

7. The syringe of claim 6 wherein said filter element is disposed in the longer of said first and second lumens.

8. The syringe of claim 1 wherein said first chamber is adapted to hold a plurality of microparticles disposed in a suspension medium and said filter element has pores smaller than the average size of said plurality of microparticles.

9. A method of delivering microparticles to a target space through the syringe of claim 1 comprising the steps of:
a. placing a plurality of microparticles in a suspension medium within said first chamber;
b. activating said first plunger to create a positive pressure within said first chamber to force said microparticle suspension through said first lumen of said needle to said target space;
c. activating said second plunger to create a negative pressure within said second chamber to draw said suspension medium through said second lumen of said needle and said filter element into said second chamber;
d. wherein said filter element has pores smaller than the average size of said microparticles such that said microparticles are separated from the suspension medium and left in said target space when said suspension medium is drawn into said second chamber.

10. The method of claim 9 wherein steps b and c are repeated multiple times.

11. A microparticle delivery syringe comprising:
a. at least a fluid chamber;
b. at least a plunger respectively disposed in said fluid chamber;
c. a needle comprising at least a lumen in fluid communication with said fluid chamber; and
d. a filter element disposed in the lumen.

12. A method of delivering microparticles to a target space comprising the steps of:
a. inserting a trocar with a guide channel into a target space such that said guide channel is in fluid communication with said target space;
b. placing a plurality of microparticles in a suspension medium within a conventional syringe and injecting said microparticles in said suspension medium through said guide channel into said target space;
c. placing the syringe of claim 11 in said guide channel and activating said plunger to create a negative pressure with said fluid chamber to draw said suspension medium through said lumen of said needle and said filter element into said fluid chamber;
d. wherein said filter element has pores smaller than the average size of said microparticles such that said microparticles are separated from the suspension medium and left in said target space when said suspension medium is drawn into said fluid chamber.

13. A hypodermic needle comprising:
a shaft comprising at least a lumen;
a filter disposed at a distal end of the shaft; and
at least a connector disposed at the proximal end of the shaft for fluidly connecting the lumen to a syringe.

14. The hypodermic needle of claim 13 wherein the shaft comprises a plurality of lumens fluidly isolated from each other and the filter is disposed at the distal end of one of the plurality of lumens.

15. The hypodermic needle of claim 14 wherein the filter is disposed on or within the longest of the plurality of lumens.

16. The hypodermic needle of claim 14 further comprising a plurality of connectors.

16. The hypodermic needle of claim 13 wherein the filter is disposed flush with the distal opening of the lumen.

17. The hypodermic needle of claim 13 wherein the filter is provided by an insert disposed within the lumen.

18. The hypodermic needle of claim 13 wherein the filter is provided by a plurality of holes in at least a sidewall of the lumen.

19. The syringe of claim 1 wherein the shaft has at least a wall that fluidically isolates the at least two lumens from each other along the entire length of the shaft.

20. A microparticle delivery syringe for delivering a microparticle suspension to a target space, the microparticle delivery syringe comprising:
a. a first chamber;
b. a second chamber fluidly separated from said first chamber;
c. a needle having a shaft with at least two lumens, the shaft having at least a wall that fluidically isolates the at least two lumens from each other along the entire length of the shaft. wherein one of said lumens is in fluid communication with said first chamber and wherein the other of said lumens is in fluid communication with said second chamber; and
d. a filter element disposed at a distal end of the shaft in one of said lumens.
